(19) United States
(12) Patent Application Publication
(10) Pub. No.: US 2009/0326345 A1
(43) Pub. Date: Dec. 31, 2009

(54) RESULT VALIDATION IN NON-INVASIVE CEREBRAL OXYGENATION LEVEL MONITORING

(75) Inventors: Richard A. Jaffe, Palo Alto, CA (US); Jaime R. Lopez, El Granada, CA (US); Xuefeng Cheng, Cupertino, CA (US)

Correspondence Address: COOPER & DUNHAM, LLP, 30 Rockefeller Plaza, 20th Floor, NEW YORK, NY, 10112, US

(73) Assignee: O2 MEDTECH, INC.

(21) Appl. No.: 12/146,754

(22) Filed: Jun. 26, 2008

(51) Int. Cl. A61B 5/1455

(52) U.S. Cl. 600/323

(57) ABSTRACT

Methods, systems, and related computer program products for optically monitoring a chromophore level in a body part of a patient are described. An optical source introduces optical radiation into the body part, and an optical detector receives optical radiation that has propagated through at least a portion of the body part and produces a first signal representative of the received optical radiation. The first signal is processed to produce a chromophore level metric, which is output on a user display, and is further processed to produce a second signal known to exhibit measurably significant timewise fluctuations corresponding to at least one intrinsic physiological oscillation of the patient when the optical source and the optical detector are in proper optical coupling with the body part. An error condition indication is provided if the measurably significant timewise fluctuations are not present in the second signal.

### FIELD

This patent specification relates generally to the measurement of chromophore concentrations or other properties of biological tissue using information acquired from non-invasive optical scans thereof. More particularly, this patent specification relates to cerebral oxygenation level monitoring using near-infrared (NIR) optical scanning.

### BACKGROUND AND SUMMARY OF THE DISCLOSURE

The use of near-infrared light as a basis for the measurement of biological properties or conditions in living tissue is particularly appealing because of its relative safety as compared, for example, to the use of ionizing radiation. Various techniques have been proposed for non-invasive near-infrared spectroscopy of biological tissue. Generally speaking, these techniques are directed to detecting the presence and/or measuring the concentrations of one or more chromophores in the biological tissue, such as blood hemoglobin in oxygenated (HbO) and deoxygenated (Hb) states.

One exemplary need for fast, safe imaging of chromophore concentrations in biological tissue, particularly oxygenated hemoglobin levels for the human brain, arises in the context of the millions of surgical procedures performed under general anesthesia every year. One statistic recited in U.S. Pat. No. 5,902,235 is that at least 2,000 patients die each year in the United States alone due to anesthetic accidents, while numerous other such incidents result in at least some amount of brain damage. As discussed therein, certain surgical procedures, particularly of a neurological, cardiac or vascular nature, may require induced low blood flow or pressure conditions, which inevitably involves the potential of insufficient oxygen delivery to the brain. At the same time, the brain is highly intolerant to oxygen deprivation, and brain cells will die within a few minutes if not sufficiently oxygenated. Accordingly, the availability of immediate and accurate information concerning brain oxygenation levels is of critical importance to anesthesiologists and surgeons, as well as other involved medical practitioners. Hospital emergency rooms represent another real-world need for fast, safe measurement of brain oxygenation levels.

Pulse oximetry, in which infrared sources and detectors are placed across a thin part of the patient's anatomy such as a fingertip or earlobe, has arisen as a standard of care for all operating room procedures. However, pulse oximetry provides only a general measure of blood oxygenation as represented by the blood passing by the fingertip or earlobe sensor, and does not provide a measure of oxygen levels in vital organs such as the brain. In this sense, the surgeons in the operating room essentially “fly blind” with respect to brain oxygenation levels, which can be a major source of risk for patients (e.g., stroke) as well as a major source of cost and liability issues for hospitals and medical insurers.

As used herein, near-infrared (NIR) cerebral oxygenation level monitoring refers to the transcranial introduction of NIR radiation (e.g., in the 500-1000 nm range) into the intracranial compartment and the processing of received NIR radiation migrating outward therefrom to generate at least one metric indicative of oxygenation level(s) in the brain. One example of an oxygenation level metric is oxygen saturation, which refers to the fraction or percentage of total hemoglobin [HbT] that is oxygenated hemoglobin [HbO]. Depending on the particular NIR measurement strategy used, the oxygen saturation can be “relative” in nature (ie., presented only in terms of its change over time) or, more preferably, can be “absolute” in nature (i.e., computed from absolute concentrations of [HbO] and [HbT] in units of grams per deciliter (g/dl) or equivalent). Another example of an oxygenation level metric would be the oxygenated hemoglobin concentration [HbO], which can be provided in absolute or relative terms depending on the NIR measurement strategy used. Other quantities for oxygenation level metrics can also be used as would be apparent to a person skilled in the art.

Valid NIR cerebral oxygenation level readings provide crucial monitoring data for the surgeon and other attending medical personnel, providing more direct data on brain oxygenation levels than pulse oximeters while being just as safe and non-invasive as pulse oximeters. Examples of systems for NIR cerebral oxygenation level monitoring are discussed in the following references, each of which is incorporated by reference herein: U.S. Pat. No. 4,972,331, U.S. Pat. No. 5,119,815, U.S. Pat. No. 5,187,672, U.S. Pat. No. 5,386,827, U.S. Pat. No. 6,526,309, U.S. Pat. No. 5,902,235, U.S. Pat. No. 7,047,054, and US 2006/0015021A1. Generally speaking, such systems involve the attachment of an NIR probe patch, or multiple such NIR probe patches, to the forehead and/or other available skin surface of the head. Each NIR probe patch usually comprises one or more NIR optical sources for introducing NIR radiation into the cerebral tissue and one or more NIR optical receivers for detecting NIR radiation that has migrated through at least a portion of the cerebral tissue. One or more oxygenation level metrics are then provided on a viewable display in a digital readout and/or graphical format.

However, due to particular combinations of technological and environmental factors that can exist in real-world NIR cerebral oxygenation level monitoring system implementations, difficulties can arise in ensuring the validity of the oxygenation level metrics provided thereby. These factors can include, but are not necessarily limited to, (i) the presence of ambient NIR radiation sources in the clinical setting (e.g., lighting, heating, instrumentation systems), (ii) the relatively high sensitivity of the NIR detectors on the probe patches as necessitated by the high lossiness of the skull and cerebral tissue, and (iii) natural absorptances and/or reflectivities in the NIR spectrum of various physical things present in the clinical setting (e.g., hospital bedding, clothing, plastics, etc.). Due to these and possibly other factors, there exists an undesirable possibility that an NIR cerebral oxygenation level monitoring system may provide “normal” readings even though there is a coupling failure between the NIR probe patch and the skin surface of the patient. In one extreme scenario, the NIR probe patch may even fall completely off the patient, but due to the peculiar combinations mentioned above, just the right amount of NIR radiation might be entering the NIR detectors on the NIR probe patch such that a “normal” reading is provided by the NIR cerebral oxygenation level monitoring system.

The above coupling failure scenarios, the most extreme of which might be called the “living gurney” scenario, can have potentially catastrophic consequences. For example, there may be a situation where a surgical patient is being monitored by an ECG system, a pulse oximeter system, and an NIR cerebral oxygenation level monitoring system The NIR probe patch may have become accidentally decoupled from the patient, but the NIR cerebral oxygenation level monitoring system has continued to provide “normal” readings. In the meantime, the patient has an ischemic event in which actual cerebral tissue oxygenation levels fall precipitously low. This ischemic event will be transparent to the ECG system because the patient's heart rate is fine, and will further be transparent to the pulse oximeter system because the peripheral arterial blood oxygenation at the patient's finger is fine. Because the ischemic event has not been picked up by the NIR cerebral oxygenation level monitoring system, it may go undetected until it is too late and permanent brain damage has occurred, such as with a stroke. Other issues arise as would be apparent to a person skilled in the art in view of the present disclosure.

Described in this patent specification are methods, systems, and related computer program products for NIR cerebral oxygenation level monitoring with improved result validation. According to one preferred embodiment, a method for NIR cerebral oxygenation level monitoring is provided, comprising causing an NIR optical source to introduce NIR optical radiation into the cerebral tissue of a patient for migration toward an NIR optical detector, and comprising receiving a first signal representative of NIR optical radiation detected by the NIR optical detector. The first signal is processed to produce a cerebral oxygenation level metric, and the cerebral oxygenation level metric is then output onto a user display. The method further comprises processing the first signal to generate a second signal known to exhibit measurably significant timewise fluctuations corresponding to at least one intrinsic physiological oscillation of the patient when the NIR optical source and the NIR optical detector are in proper optical coupling with the cerebral tissue. The method further comprises detecting whether such measurably significant timewise fluctuations are indeed present in the second signal, and outputting an indication of an error condition if the measurably significant timewise fluctuations are not present in the second signal. Examples of intrinsic physiological oscillations include intrinsic respiratory oscillations and cardiac oscillations. Preferably, the indication of the error condition comprises one or more of (i) preventing the outputting of the cerebral oxygenation level metric on the user display, (ii) outputting a visual error indicator on the user display in viewable conjunction with the oxygenation level metric, and (iii) sounding an audible alarm.

According to another preferred embodiment, a system for NIR cerebral oxygenation level monitoring is provided, comprising an NIR optical source for introducing NIR optical radiation into the cerebral tissue of a patient. The system further comprises an NIR optical detector for receiving NIR optical radiation that has propagated through at least a portion of the cerebral tissue and for producing a first signal representative of the received NIR optical radiation. The system further comprises a first processor configured and adapted to process the first signal to produce a cerebral oxygenation level metric therefrom, and a user display for displaying the cerebral oxygenation level metric. The first processor is also configured and adapted to produce a second signal from the first signal, wherein the second signal is known to exhibit measurably significant timewise fluctuations corresponding to at least one intrinsic physiological oscillation of the patient when the NIR optical source and the NIR optical detector are in proper optical coupling with the cerebral tissue. The system further comprises a second processor configured and adapted to detect whether such measurably significant timewise fluctuations are indeed present in the second signal, and to cause the user display to indicate an error condition if the measurably significant timewise fluctuations are not present in the second signal.

### BRIEF DESCRIPTION OF THE DRAWINGS

FIG. 1 illustrates a system for near-infrared (NIR) cerebral oxygenation level monitoring according to a preferred embodiment;

FIG. 2 illustrates a system for NIR cerebral oxygenation level monitoring according to a preferred embodiment; and

FIG. 3 illustrates improved result validation in NIR cerebral oxygenation level monitoring according to a preferred embodiment.

### DETAILED DESCRIPTION

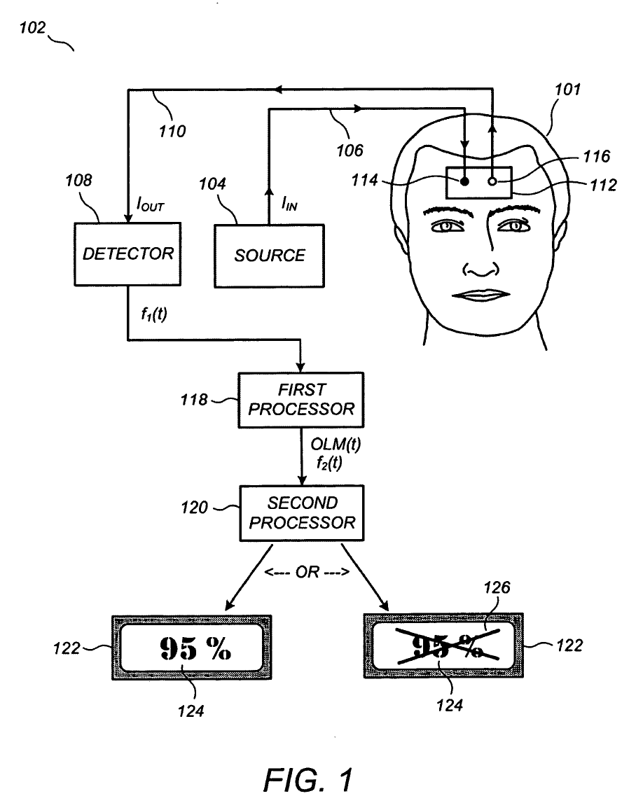

FIG. 1 illustrates a system 102 for near-infrared (NIR) cerebral oxygenation level monitoring according to a preferred embodiment. Spectrophotometric systems based on visible and/or near infrared (NIR) radiation for achieving various non-invasive physiological measurements, such as cerebral oxygenation level measurements, have been in various stages of proposal and development for an appreciable number of years. As will be appreciated by one skilled in the art in view of the present disclosure, certain component devices suitable for use within the system 102 are described in several prior disclosures directed to such oxygenation level measurements and their specifics will not be re-described here. Moreover, several of those overall spectrophotometric systems and methods may be advantageously used in conjunction with, or as important components within, a system 102 according to one or more of the preferred embodiments. Examples of such spectrophotometric systems include, but are not limited to: continuous wave spectrophotometers (CWS) as discussed in WO1992/20273A2 and WO1996/16592A1; phase modulation spectroscopic (PMS) units as discussed in U.S. Pat. No. 4,972,331, U.S. Pat. No. 5,187,672, and WO1994/21173A1; time resolved spectroscopic (TRS) units as discussed in U.S. Pat. No. 5,119,815, U.S. Pat. No. 5,386,827, and WO1994/22361A1; and phased array systems as discussed in WO1993/25145A1. All of the patents and patent publications identified above in this paragraph are incorporated by reference herein. Another example of a spectrophotometric system that is particularly suitable for use in conjunction with one or more of the preferred embodiments is discussed in US 2006/0015021A1, which is also incorporated by reference herein.

System 102 comprises an optical source 104 that emits radiation having a wavelength in the range of about 500 nm-1000 nm, i.e., in the upper visible and near infrared wavelengths. Light from the optical source 104 is carried by an optical fiber 106 to a source port 114 of an optical coupling device 112 on the forehead of the patient. Light that has migrated through at least a portion of the cerebral tissue and outward again is collected at a detection port 116 of the optical coupling device 112 and guided to an optical detector 108 by an optical fiber 110. For one preferred embodiment, the optical coupling device 112 can be similar to one or more of the optical coupling devices disclosed in U.S. Pat. No. 5,596,987, which is incorporated by reference herein. Preferably, the optical coupling device 112 is designed to be a disposable, one-time-use patch that secures to the forehead using known adhesives. The optical coupling device 112 including the source port 114 and detection port 116 can alternatively be attached to an accessible skin surface elsewhere on the scalp other than the forehead.

The detector 108 generates a first signal f₁(t) that is representative of the light collected at the detection port 116. For a relatively simple continuous wave embodiment in which the source 104 emits a monochromatic unmodulated carrier wave, the first signal f₁(t) can be a voltage signal representing an instantaneous intensity of the light collected at detection port 116. For one embodiment, the optical source 104 comprises a 4 mW laser diode emitting at 760 nm, and the optical detector 108 comprises a Hamamatsu R928 photomultiplier tube. Although the optical source 104, optical detector 108, and optical coupling device 112 are illustrated as distinct components in the example of FIG. 1, the scope of the present teachings is not so limited. For example, in other preferred embodiments, the optical source(s) and optical detector(s) can be integrated into a single patch that adheres to the skin surface, such that there is no need for external optical connections to the adhesive patch assembly. Any of a variety of other schemes for causing optical radiation to be introduced into the cerebral tissue and for causing optical radiation propagating back out of the cerebral tissue to be detected can be used without departing from the scope of the preferred embodiments.

System 102 further comprises a first processor 118 that receives the first signal f₁(t) and performs processing to generate therefrom a cerebral oxygenation level metric OLM(t). The particular type of oxygenation level metric OLM(t) that is provided will depend upon the particular type of spectrophotometric scheme in use. As known in the art, expected values for oxygen saturation level metrics will usually lie in the 70%-100% range, while expected values for oxygenated hemoglobin concentration will usually lie somewhere between a dangerously low level of 7 grams per deciliter (g/dl) (or less) and a dangerously high level of about 17 g/dl (or more), and usually within a much narrower sub-interval thereof depending on patient characteristics and the medical procedure taking place. The oxygenation level metric OLM(t) is displayed on a user display 122. For the example of FIG. 1, OLM(t) is presumed to be an oxygen saturation metric and is shown as a digital readout 124 on the user display 122, although any of a variety of different digital and/or graphical output schemes can be used.

However, for at least the reasons described supra, and as best exemplified by the frightening “living gurney” scenario, the computed values of OLM(t) will not necessarily be self-validating. In other words, the mere fact that the computed values for OLM(t) may fall somewhere in their “normal” ranges does not necessarily mean that reliable monitoring of OLM(t) has, in fact, taken place. It has been found, however, that at least one additional cross-check or validation can be performed on the detected spectrophotometric information that can avoid catastrophic false negative scenarios. In particular, regardless of the particular type of spectrophotometric scheme in use, it has been found that there will often exist at least one signal somewhere in the chain of computation for OLM(t), or alternatively some signal that can be derived from signals in that chain of computation, that will exhibit measurably significant timewise fluctuations corresponding to at least one intrinsic physiological oscillation in the patient when the NIR optical source and the NIR optical detector are in proper optical coupling with the cerebral tissue, and, conversely, that will fail to exhibit such measurably significant fluctuations if there has been a coupling error such as the NIR probe patch falling off the patient.

As used herein, intrinsic physiological oscillation refers to a physiological characteristic or behavior that is brought about autonomically by the patient's body and that exhibits some form of periodicity. One example of an intrinsic physiological oscillation is the patient's intrinsic respiratory oscillations, i.e., their natural breathing, which generally occurs at a periodic rate somewhere between 3 breaths per minute (0.05 Hz) and 30 breaths per minute (0.5 Hz). Another example of an intrinsic physiological oscillation is the patient's cardiac oscillations, which generally occur at a rate somewhere between 30 beats per minute (0.5 Hz) to 180 beats per minute (3 Hz). The oscillation referred to in this patent specification preferably is substantially periodic although the frequency can drift and even change abruptly, and in certain embodiments the term as used in this patent specification encompasses measurably significant timewise fluctuations due to patient actions that are not consistently periodic, such as, for example, breathing that may stop for a short period and resume with breaths that are not at regular intervals. Thus, in certain preferred embodiments the term oscillation refers to changes due to events that are generally periodic, although the period can fluctuate, and in other embodiments it refers to changes that may not be entirely periodic.

The specific identity of the particular signal known to exhibit measurably significant timewise fluctuations corresponding to the at least one intrinsic physiological oscillation in the patient, that particular signal being denoted herein as a second signal f₂(t), will often depend upon the specific type of spectrophotometric scheme in use. The second signal f₂(t) might be identified, for example, as a certain attenuation coefficient pa(t) that is computed somewhere in the “guts” of the algorithm from which the output value OLM(t) is computed. In other cases, the second signal f₂(t) could be a certain computed phase delay for PMS spectrophotometric schemes. In other cases, the second signal f₂(t) could be an intermediate quantity (an certain eigenvalue of a certain intermediate matrix, for example) whose physical significance is less readily apparent, but which is known, either empirically or analytically, to oscillate with the cardiac, respiratory, and/or other intrinsic oscillatory cycle of the patient. In still other cases, the second signal f₂(t) could directly correspond to the first signal f₁(t), as may be the case for CWS spectrophotometric schemes, and in still other cases the second signal f₂(t) may even be the signal OLM(t) itself for still other types of spectrophotometric schemes. As a function of the particular NIR spectrophotometric scheme being used, the particular identity for the second signal f₂(t) could be determined analytically and/or empirically by a person skilled in the art in view of the present disclosure without undue experimentation.

Using cardiac oscillations as an example of an intrinsic physiological oscillation in the patient, in order to exhibit measurably significant timewise fluctuations corresponding to cardiac oscillations, it is not required that the second signal f₂(t) be a purely AC signal at the cardiac frequency, or even to have a particularly large AC component at the cardiac frequency relative to the DC component and/or other non-cardiac frequency components. By way of example, the second signal f₂(t) might have a relatively large DC component, and may have an AC component at the cardiac frequency that is only one percent or even a fraction of a percent of the DC component, but as long as that cardiac component can be extracted in a measurably significant way (for example, by having a peak-to-peak or RMS value that is greater than a predetermined threshold), then it can be concluded that the second signal f₂(t) exhibits a measurably significant timewise fluctuation corresponding to the cardiac oscillations of the patient.

As illustrated in FIG. 1, according to a preferred embodiment, the first processor 118 processes the first signal f₁(t) to extract the second signal f₂(t) as well to generate the oxygenation level metric OLM(t). Depending on the identity of the second signal f₂(t), the processing stream or thread that extracts f₂(t) can be the same as, partially overlapping with, or entirely separate than the processing stream or thread that generates the oxygenation level metric OLM(t). The functionality of the first processor 1 18 can be broken up among as many distinct processors as may be desired or required. System 102 further comprises a second processor 120 (which can be optionally combined with first processor 118) configured to detect whether the measurably significant timewise fluctuations are present in the second signal f₂(t). If such measurably significant timewise fluctuations are not present, then an error condition is indicated. In the example of FIG. 1, the error condition is indicated by virtue of a large error marker 126 placed in viewable conjunction with (e.g., superimposed upon) the digital readout 124. In other preferred embodiments, the error condition can be indicated by simply preventing the digital readout 124 altogether (for example, showing dash characters “- - - ” instead of the OLM(t) metric), and/or by sounding an audible alarm. In still other preferred embodiments, the value of the digital readout 124 can still be shown, but with a noticeable nearby readout of the phrase “CHECK SENSOR” and/or other words or phrases to put the viewer on notice of a potentially unreliable reading. Any of a variety of other methods for communicating an error condition to medical personnel can be used without departing from the scope of the preferred embodiments.

The detection by the second processor 120 of whether there is a measurably significant timewise fluctuation in the second signal f₂(t) according to at least one intrinsic physiological oscillation can be performed in any of a variety of different ways without departing from the scope of the preferred embodiments. For one preferred embodiment in which the at least one intrinsic physiological oscillation is selected to consist only of the cardiac oscillations of the patient, the second signal f₂(t) is filtered by a bandpass filter having a passband of 0.5 Hz-3 Hz and then thresholded, because the cardiac oscillations (heartbeat) of the patient usually occur at a rate somewhere between 30 beats per minute (0.5 Hz) to 180 beats per minute (3 Hz). For another preferred embodiment in which the at least one intrinsic physiological oscillation is selected to consist only of the respiratory oscillations of the patient, the second signal f₂(t) is filtered by a bandpass filter having a passband of 0.05 Hz-0.5 Hz and then thresholded, because the respiratory oscillations of the patient usually occur at a rate somewhere between 3 breaths per minute (0.05 Hz) and 30 breaths per minute (0.5 Hz). In still another preferred embodiment in which both the respiratory and cardiac oscillations are considered, the bandpass filter may extend between 0.05 Hz and 3 Hz, and different sub-intervals therein may optionally be weighted differently if one or the other of the respiratory and cardiac oscillations is known to appear more prominently in the second signal f₂(t). The speed of the hardware for the first processor 118 should be sufficiently fast to compute sample points for f₂(t) at a rate that is sufficient to reliably include the intrinsic physiological oscillations being detected. For example, in order to reliably include a 3 Hz cardiac oscillation, the first processor 118 should be able to compute the signal f₂(t) at least six times per second, and preferably much faster for even better inclusion and detection.

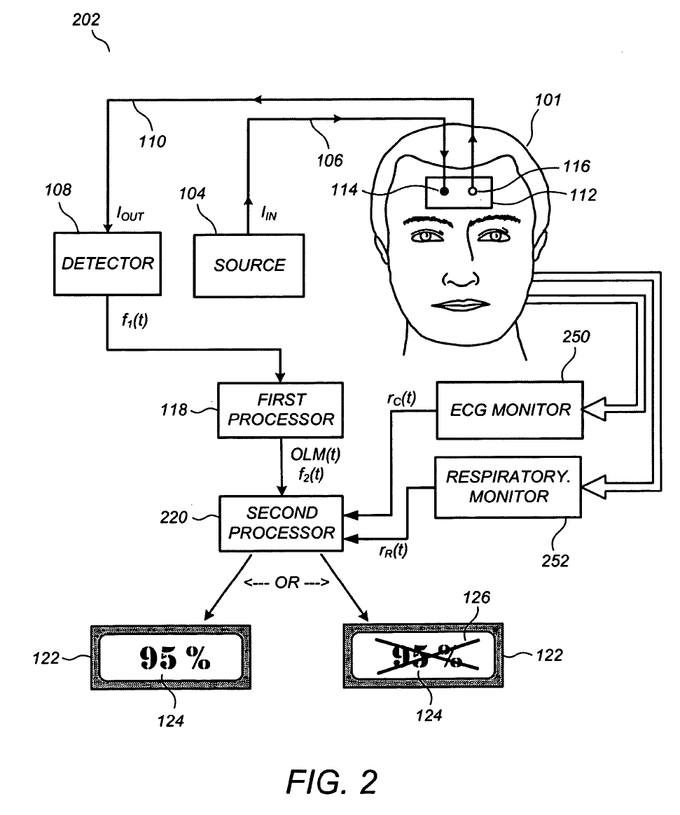

FIG. 2 illustrates a system 202 for near-infrared (NIR) cerebral oxygenation level monitoring according to a preferred embodiment that is similar to the system 102 of FIG. 1, supra, except that a second processor 220 thereof is programmed to receive both (i) the second signal f₂(t) and (ii) at least one externally measured version of the intrinsic physiological oscillation whose presence is to be detected in the second signal f₂(t), and then to use that externally measured version as a reference signal in a lock-in detection scheme for extracting that intrinsic physiological oscillation from the second signal f₂(t). Thus, by way of example, FIG. 2 includes an external ECG monitor 250 providing a cardiac signal r_{C}(t) to the second processor 120. The external ECG monitor 250 is coupled to the patient in a conventional manner using physical ECG interfaces generally unrelated to the optical coupling device 112. The second processor 220 is configured and adapted to detect the presence of a cardiac oscillation in the second signal f₂(t) using a lock-in detection scheme in which the cardiac signal r_{C}(t) serves as a reference signal.

As used herein, lock-in detection refers to receiving an input signal and a periodic reference signal and synchronously extracting frequency component(s) from the input signal that correspond to the frequency content of the periodic reference signal. Generally speaking, where a periodic reference signal is made available, lock-in detection is highly superior to passive bandpass filtering with respect to signal-to-noise performance, providing an ability to detect relatively faint periodic components even in a relatively noisy environment. Although the preferred embodiment of FIG. 2 adds an additional layer of complexity to that of FIG. 1, it is often the case that there will already be an ECG connected to the patient in the ordinary course of the procedure. The use of such synchronous detection may be required if f₂(t) is too noisy for reliable passive detection of a cardiac oscillatory component therein. The use of such synchronous detection can be advantageous by providing increased certainty that the oscillatory component being extracted from f₂(t) really does, in fact, correspond to the cardiac oscillations of this patient and not to some other environmental oscillation in the room that is between 0.5 Hz-3 Hz that might be finding its way somehow into f₂(t), thereby even further reducing the possibility of false negative ischemic conditions.

Also illustrated in FIG. 2 is an external respiratory monitor 252 providing a respiratory signal r_{R}(t) to the second processor 220. The external respiratory monitor 252 is also coupled to the patient in a conventional manner using respiratory sensor interfaces generally unrelated to the optical coupling device 112. For one preferred embodiment, the second processor 220 is further configured and adapted to detect the presence of a respiratory oscillation in f₂(t) using a lock-in detection scheme in which the respiratory signal r_{R}(t) serves as a reference signal, wherein the measurably significant presence of both cardiac and respiratory components in f₂(t) is required to validate the output readings (i.e., to establish that there is no optical coupling failure between the optical coupling device 112 and the cerebral tissue of the patient). In other preferred embodiments, only one or the other of the external cardiac monitor 250 and external respiratory monitor 252 is used, and only the measurably significant presence in f₂(t) of the cardiac or respiratory component, respectively, is required to validate the output readings. In still another preferred embodiment, the system is adapted and configured to be user-switchable, such that the user may choose one of the externally measured signals r_{C}(t) and r_{R}(t) to be used, or may choose that both of them be used. In yet another preferred embodiment, the user may choose that neither of the externally measured signals r_{C}(t) and r_{R}(t) is to be used, in which case the second processor 220 will automatically revert to the passive bandpass filtering scheme of FIG. 1.

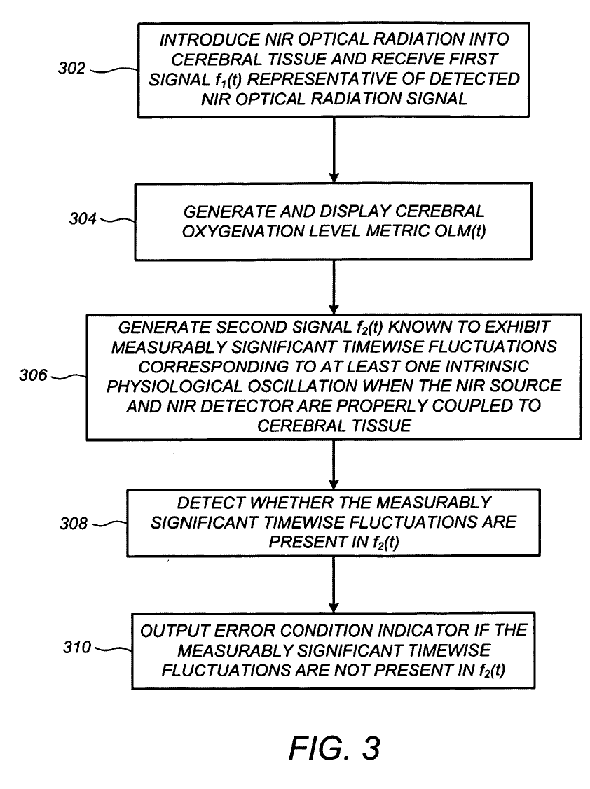

FIG. 3 illustrates improved result validation in NIR cerebral oxygenation level monitoring according to a preferred embodiment. At step 302, NIR optical radiation is introduced into the cerebral tissue from the NIR source(s), and NIR radiation is detected by the NIR optical detector(s) to provide the first signal f₁(t). At step 304, a cerebral oxygenation level metric OLM(t) is generated from the first signal f₁(t) and displayed. At step 306, a second signal f₂(t) is generated that is known to exhibit measurably significant timewise fluctuations according to at least one intrinsic physiological oscillation of the patient when the NIR optical source and the NIR optical detector are in proper optical coupling with the cerebral tissue. For one preferred embodiment, the step 306 can be performed entirely separately from the step 304 using only the “raw” received NIR signals. In other preferred embodiments, the step 306 may be integral with the step 304, such as when a particular beginning, intermediate, or ultimate variable in the computation of OLM(t) meets the criteria for the second signal f₂(t). At step 308, it is detected whether the second signal f₂(t) contains the measurably significant timewise fluctuations. As discussed supra, according to one preferred embodiment, the step 308 comprises passive bandpass filtering to detect the presence of the measurably significant timewise fluctuations, while in another preferred embodiment there are externally measured reference signals provided, such as cardiac and/or respiratory signals, that are used to synchronously detect the presence of the measurably significant timewise fluctuations.

Whereas many alterations and modifications of the preferred embodiments will no doubt become apparent to a person of ordinary skill in the art after having read the foregoing description, it is to be understood that the particular embodiments shown and described by way of illustration are in no way intended to be considered limiting. By way of example, the above-described features and advantages of the preferred embodiments can be applied in any of a variety of medical contexts in which any of a variety of chromophores are optically monitored in any of a variety of different body parts (e.g., kidney, lung, liver, arm, neck, etc.), such chromophores including, but not limited to, oxygenated hemoglobin, deoxygenated hemoglobin, carbamino hemoglobin, carboxymethylated hemoglobin, glucose, cytochromes, cytosomes, cytosols, enzymes, hormones, neurotransmitters, chemical or chemotransmitters, proteins, cholesterols, apoproteins, lipids, carbohydrates, and dyes or other contrast agents. By way of further example, although described supra in terms of near-infrared (NIR) optical wavelengths in the 500-1000 nm range, the features and advantages of the preferred embodiments are applicable for a wider range of NIR optical wavelengths between 500-2500 nm, as well as for other optical radiation wavelengths in the ultraviolet, visible, and infrared ranges.

By way of further example, within the scope of the preferred embodiments is a system for optically monitoring a chromophore level in a body part of a patient, comprising an optical source for introducing optical radiation into the body part. The system further comprises an optical detector for receiving optical radiation that has propagated through at least a portion of the body part and for producing a first signal representative of the received optical radiation. The system further comprises a first processor configured and adapted to process the first signal to produce a chromophore level metric therefrom, and a user display for displaying the chromophore level metric or other medical property computed from the chromophore level metric. The first processor is also configured and adapted to produce a second signal from the first signal, wherein the second signal is known to exhibit measurably significant timewise fluctuations corresponding to at least one intrinsic physiological oscillation of the patient when the optical source and the optical detector are in proper optical coupling with the body part. The system further comprises a second processor configured and adapted to detect whether such measurably significant timewise fluctuations are indeed present in the second signal, and to cause the user display to indicate an error condition if the measurably significant timewise fluctuations are not present in the second signal. By ensuring the presence of certain physiologically-related frequency components somewhere in the computational chain of the property being optically monitored, an optical monitoring system according to one or more of the preferred embodiments provides increased confidence that the current “normal” readout is resulting from a truly “normal” level for that property rather than from a fluke (e.g., “living gurney”) scenario in which the optical monitoring probe has become disconnected but in which ambient conditions still cause a “normal” readout. Therefore, reference to the details of the preferred and other embodiments are not intended to limit their scope, which is limited only by the scope of the claims set forth below.

What is claimed is:

1. A method for near-infrared (NIR) cerebral oxygenation level monitoring, comprising:
causing an NIR optical source to introduce NIR optical radiation into the cerebral tissue of a patient for migration toward an NIR optical detector;
receiving a first signal representative of the NIR optical radiation detected by the NIR optical detector;
processing the first signal to produce a cerebral oxygenation level metric and outputting the cerebral oxygenation level metric on a user display;
processing the first signal to generate a second signal therefrom known to exhibit measurably significant timewise fluctuations corresponding to at least one intrinsic physiological oscillation in the patient when said NIR optical source and said NIR optical detector are in proper optical coupling with said cerebral tissue;
detecting whether said measurably significant timewise fluctuations are present in said second signal; and
outputting an indication of an error condition noticeable to a viewer of the user display if said measurably significant timewise fluctuations are not present in said second signal.

2. The method of claim 1, wherein said outputting said indication of the error condition comprises at least one of (i) preventing said outputting of the cerebral oxygenation level metric on the user display, (ii) outputting a visual error indicator on the user display in viewable conjunction with the oxygenation level metric, and (iii) sounding an audible alarm.

3. The method of claim 1, wherein said at least one intrinsic physiological oscillation is selected from the group consisting of intrinsic cardiac oscillations and intrinsic respiratory oscillations.

4. The method of claim 1, wherein said detecting whether the measurably significant timewise fluctuations are present comprises passively filtering said second signal using a bandpass filter corresponding to a frequency range of said intrinsic physiological oscillations.

5. The method of claim 1, wherein said at least one intrinsic physiological oscillation includes an intrinsic cardiac oscillation, and wherein said detecting whether said measurably significant timewise fluctuations are present in said second signal comprises passively filtering said second signal with a bandpass filter extending from about 0.5 Hz-3 Hz.

6. The method of claim 5, wherein said detecting whether said measurably significant timewise fluctuations are present in said second signal further comprises thresholding the bandpass-filtered second signal with a predetermined threshold value.

7. The method of claim 1, wherein said at least one intrinsic physiological oscillation includes an intrinsic respiratory oscillation, and wherein said detecting whether said measurably significant timewise fluctuations are present in said second signal comprises passively filtering said second signal with a bandpass filter extending from about 0.05 Hz-0.5 Hz and assessing a magnitude of the bandpass-filtered second signal.

8. The method of claim 1, wherein said at least one intrinsic physiological oscillation includes both an intrinsic cardiac oscillation and an intrinsic respiratory oscillation, and wherein said detecting whether said measurably significant timewise fluctuations are present in said second signal comprises:
extracting a cardiac component of the second signal having a frequency range between about 0.5-3 Hz; extracting a respiratory component of the second signal having a frequency range between about 0.05-0.5 Hz; and
thresholding a weighted sum of said cardiac and respiratory components with a predetermined threshold.

9. The method of claim 1, wherein said detecting whether said measurably significant timewise fluctuations are present in said second signal comprises:
receiving a third signal indicative of an externally measured version of said at least one intrinsic physiological oscillation in the patient; and
synchronously detecting said measurably significant timewise fluctuations in said second signal using said third signal as a reference signal.

10. The method of claim 9, wherein said at least one intrinsic physiological oscillation includes an intrinsic cardiac oscillation, and wherein said third signal is acquired using an external cardiac monitor that is functionally separate from said NIR optical source and said NIR optical detector.

11. The method of claim 9, wherein said at least one intrinsic physiological oscillation includes an intrinsic respiratory oscillation, and wherein said third signal is acquired using an external respiratory monitor that is functionally separate from said NIR optical source and said NIR optical detector.

12. A system for near-infrared (NIR) cerebral oxygenation level monitoring, comprising:
an NIR optical source for introducing NIR optical radiation into the cerebral tissue of a patient;
an NIR optical detector for receiving NIR optical radiation that has migrated through at least a portion of the cerebral tissue, said NIR optical detector generating a first signal representative of the received NIR optical radiation;
a first processor configured and adapted to process the first signal to generate a cerebral oxygenation level metric therefrom, said first processor being further configured and adapted to generate a second signal from the first signal, the second signal being known to exhibit measurably significant timewise fluctuations corresponding to at least one intrinsic physiological oscillation of the patient when said NIR optical source and said NIR optical detector are in proper optical coupling with the cerebral tissue;
a user display for displaying the cerebral oxygenation level metric; and
a second processor configured and adapted to detect whether the measurably significant timewise fluctuations are present in the second signal and to cause an indication of an error condition noticeable to a viewer of the user display if said measurably significant timewise fluctuations are not present in said second signal.

13. The system of claim 12, wherein causing the indication of the error condition comprises at least one of (i) causing the user display to at least partially obscure the displayed cerebral oxygenation level metric, (ii) causing the user display to display a visual error indicator in viewable conjunction with the oxygenation level metric, and (iii) causing the user display to sound an audible alarm.

14. The system of claim 12, wherein said at least one intrinsic physiological oscillation is selected from the group consisting of intrinsic cardiac oscillations and intrinsic respiratory oscillations.

15. The system of claim 12, wherein said second processor is configured and adapted to detect whether the measurably significant timewise fluctuations are present in the second signal by passively filtering said second signal using a bandpass filter corresponding to a frequency range of said intrinsic physiological oscillations.

16. The system of claim 12, wherein said second processor is configured and adapted to detect whether the measurably significant timewise fluctuations are present in the second signal by receiving a third signal indicative of an externally measured version of said at least one intrinsic physiological oscillation in the patient and synchronously detecting said measurably significant timewise fluctuations in said second signal using said third signal as a reference signal.

17. A computer program product stored on a computer-readable medium for facilitating patient monitoring, comprising:
computer code for receiving a first signal representative of near-infrared (NIR) optical radiation that has been introduced into the cerebral tissue of a patient by an NIR optical source and that has been detected by an NIR optical detector after migrating through at least a portion of the cerebral tissue theretoward;
computer code for processing the first signal to produce a cerebral oxygenation level metric for output onto a user display;
computer code for processing the first signal to generate a second signal therefrom known to exhibit measurably significant timewise fluctuations corresponding to at least one intrinsic physiological oscillation in the patient when the NIR optical source and the NIR optical detector are in proper optical coupling with the cerebral tissue;
computer code for detecting whether the measurably significant timewise fluctuations are present in the second signal; and
computer code for outputting an indication of an error condition onto the user display if said measurably significant timewise fluctuations are not present in said second signal.

18. The computer program product of claim 17, wherein the indication of the error condition comprises at least one of a visual obscuration of the cerebral oxygenation level metric, a visual error indicator near the oxygenation level metric, and an audible alarm.

19. The computer program product of claim 17, wherein said at least one intrinsic physiological oscillation is selected from the group consisting of intrinsic cardiac oscillations and intrinsic respiratory oscillations.

20. The computer program product of claim 17, wherein computer code for detecting whether the measurably significant timewise fluctuations are present in the second signal comprises:
computer code for receiving a third signal indicative of an externally measured version of said at least one intrinsic physiological oscillation in the patient; and
computer code for synchronously detecting said measurably significant timewise fluctuations in said second signal using said third signal as a reference signal.
